(19) United States
(12) Patent Application Publication
(10) Pub. No.: US 2009/0291983 A1
(43) Pub. Date: Nov. 26, 2009

(54) 3-Oxoisoindoline-1-Carboxamide Derivatives as Analgesic Agents

(75) Inventors: Yevgeni Besidski, Sodertalje (SE); Ylva Gravenfors, Sodertalje (SE); Inger Kers, Sodertalje (SE); Karin Skogholm, Sodertalje (SE); Mats Svensson, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/307,798

(22) PCT Filed: Jul. 12, 2007
(86) PCT No.: PCT/SE07/00681

§ 371 Date: Jan. 14, 2009

Related U.S. Application Data
(60) Provisional application No. 60/830,186, filed on Jul. 12, 2006.

(51) Int. Cl. A61K 31/4035; C07D 209/46; C07D 401/02; A61K 31/4439; A61P 25/00

(52) U.S. Cl. 514/339; 548/472; 546/277.1; 514/416

(57) ABSTRACT

Compounds of formula I

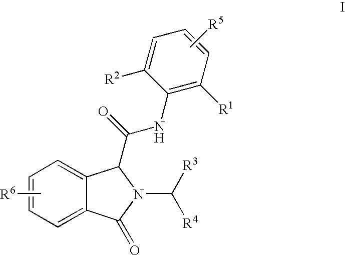

wherein R¹, R², R³, R⁴, R⁵ and R⁶ are as described in the specification, pharmaceutically acceptable salts, methods of making, pharmaceutical compositions containing and methods for using the same.

### FIELD OF THE INVENTION

This invention relates to novel pharmaceutically useful compounds, in particular compounds that are useful in the treatment of chronic, acute, neuropathic, nociceptive, visceral or inflammatory pain.

### BACKGROUND AND PRIOR ART

Voltage-gated sodium channels are critical elements in the control of electrical excitability of various cell types, including muscle and neuronal cells. In muscle and neuronal cells voltage-gated sodium channels are mainly responsible for the rising phase of the action potential (1). Voltage-gated sodium channels are composed of a single alpha subunit and one or two beta subunits (4). There are 10 known alpha subunit proteins, of which nine are functional as an ion channel (1). The different alpha subunit proteins are herein references to as NaV1.x, with x being an integer between 1 and 9. This labeling is in accordance with the conventions of the International Pharmacological Association (REF). Alpha subunits are large proteins of an approximate weight of 260 kDA (˜2000 amino acids), and are functional as voltage-gated sodium channels as monomeric structures. Four beta subunits are known at present (4). Beta subunits are smaller proteins of an approximate weight of 33-36 kDa. Beta subunits can modulate functional expression, as well as the characteristics of channel opening and closing (gating), of alpha subunits.

Five major lines of evidence support the notion that voltage-gated sodium channels are important therapeutic targets:
a) the biophysical characteristics of voltage-gated sodium channels,
b) the tissue expression pattern of voltage-gated sodium channels,
c) evidence from preclinical research,
d) the association between several congenital diseases and channelopathies of voltage-gated sodium channels, and
e) evidence from the usage of pharmacological agents active at voltage-gated sodium channels in the clinic.

A main biophysical characteristic of voltage-gated sodium channels is the fast opening and closing (activation and inactivation) of the channel upon an appropriate voltage stimulus. These features make voltage-gated sodium channels absolutely essential in the generation of the upstroke of the action potential in most neuronal and muscle cells, and thereby central to the functionality of such tissue. Thus, inhibitory pharmacological interference with the activity of NaV's is expected to have dampening effects on excitability of such tissue. Such agents may thus be useful in the treatment of diseases that involve hyperactivity of neuronal or muscle tissue.

As outlined above, there are nine functional alpha subunits of voltage-gated sodium channels. Each of these alpha subunits has a characteristic tissue expression pattern. Tissue-specific up- or down-regulation of the expression of several of the voltage-gated sodium channels in human diseases or preclinical disease models in animals strongly supports a central role for specific voltage-gated sodium channels in distinct diseases.

NaV1.7 is expressed in human neuromas, which are swollen and hypersensitive nerves and nerve endings that are often present in chronic pain states (Acta Neurochirurgica (2002) 144(8) 803-810). NaV1.7 is also expressed in dorsal root ganglion neurons and contributes to the small tetrodoxin (TTX) sensitive component seen in these cells. NaV1.7 may thus be a potential pain target in addition to its role in neuroendocrine excitability (EMBO Journal (1995) 14(6) 1084-1090).

Some 3-oxoisoindoline-1-carboxamide derivatives are known. 3-oxoisoindoline-1-carboxamide derivatives are an ideal target for multicomponent reactions (MCRs). Tetrahedron Letters (1998), 39(18), 2725-2728; Journal of Organic Chemistry (1999), 64(3), 1074-1076; and Bioorganic & Medicinal Chemistry Letters (2002), 12(14), 1813-1816 discloses some 3-oxoisoindoline-1-carboxamide derivatives prepared by so-called Ugi reactions. No pharmaceutical use of the prepared compounds is contemplated. Tetrahedron Letters (2002), 43(6), 943-946 and Journal of Organic Chemistry (2004), 69(4), 1207-1214 discloses some 3-oxoisoindoline-1-carboxamide derivatives prepared by intramolecular Diels-Alder type reactions. No pharmaceutical use of the prepared compounds is contemplated. Journal of Heterocyclic Chemistry (1997), 34(4), 1371-1374 discloses some symmetrically substituted 3-oxoisoindoline-1-carboxamide derivatives prepared by carbonylative cyclization of 2-bromobenzaldehyde with primary amines. No pharmaceutical use of the prepared compounds is contemplated. Some additional 3-oxoisoindoline-1-carboxamide derivatives are disclosed in Zhurnal Obshchei Khimii (1965), 1(7), 1292-7; Yakagaku Zasshi (1969), 89(3), 418-21; Journal of the Chemical Society, Perkin Transactions 1: Organic and Bio-Organic Chemistry (1972-1999) (1980), (4), 846-8; EP1566378; EP1661898 and CHEMCATS (Chemical Catalogs Online provided by STN); EP1566378 A1; WO03/040096; U.S. Pat. No. 5,559,256; Chemical & Pharmaceutical Bulleting (1988), 36(1), 190-201; Journal of the Chemical Society (1972-1999), (1972), (6), 835-840; Justus Liebigs Annalen Der Chemie (1978), vol 2, 283-288; Zeitschrift for Naturforschung. B, 1993, vol 48:8, 1094-1104; J. Prakt. Chem. 2, 159, 1941, 241, 244, 254; Heterocycles Vol 38; No 8; 1994, 1828-1838: J. Org. Chem. 17, 1952, 4, 8, 1-13; Tetrahedron, EN, 53, 19, 1997, 6653-6680; Tetrahedron Letters, vol 38, No 3, 1997, 359-362. EP1749817 A1 discloses isoindoline derivatives having neurogenic pain control effect.

We have surprisingly found that a novel group of 3-oxoisoindoline-1-carboxamide compounds exhibit NaV1.7 inhibiting activity, and are therefore expected to be useful in the prophylaxis and treatment of different acute and chronic pain conditions.

### DISCLOSURE OF THE INVENTION

According to the invention there is provided compounds of formula I,

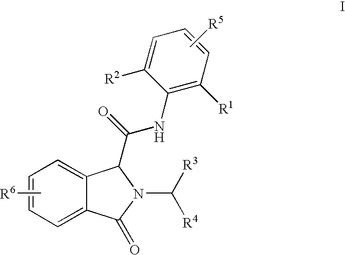

wherein
R¹ and R² represent independently, at each occurrence, halogen, C₁-C₁₂ alkyl, C₁-C₁₂ alkoxy, C₁-C₁₂ haloalkyl, C₁-C₁₂ haloalkoxy, cyano, SR⁷, N(R^{8a})R^{8b}, C₂-C₆ alkynyl, aryl or Het¹;
R³ represents hydrogen or C₁-C₁₂ alkyl;
R⁴ represents —(CH₂)ₘR⁹ or —(CH₂)ₙOR¹⁰;
further R³ and R⁴ may together represent a ring;
R⁵ represents hydrogen, C₁-C₁₂ alkyl group or C₁-C₁₂ alkoxy group (which C₁-C₁₂ alkyl and C₁-C₁₂ alkoxy groups are optionally substituted by one or more groups selected from halogen, C₂-C₆ alkenyl, C₂-C₆ alkynyl, cyano, oxo, aryl, Het⁴, —OR¹³, —SR¹⁴, —COXR¹⁵, —N(R^{16a})R^{16b}, —SO₂R¹⁷);
R⁶ represents hydrogen, C₁-C₁₂ alkyl group or C₁-C₁₂ alkoxy group (which C₁-C₁₂ alkyl and C₁-C₁₂ alkoxy groups are optionally substituted by one or more groups selected from halogen, C₂-C₆ alkenyl, C₂-C₆ alkynyl, cyano, oxo, aryl, Het⁵, —OR¹⁸, —SR¹⁹, —COXR²⁰, —N(R^{21a})R^{21b}, —SO₂R²²);
Het¹ to Het⁵ independently represent, at each occurrence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₁₋₆ alkoxy, aryl, aryloxy, —N(R^{23a})R^{23b}, —C(O)R^{23c}, —C(O)OR^{23d}, C(O)N(R^{23e})R^{23f}, —N(R^{23g})C(O)^{23h} and —N(R²³ⁱ)S(O)₂R^{23j}, OC(O)R^{23k} and a further Het;
R⁷ and R⁸ represent independently, at each occurrence, hydrogen or C₁₋₆ alkyl;
R⁹ and R¹⁰ represents aryl, Het² (which aryl and Het² optionally are substituted by one or more groups selected from halogen, C₁-C₆ alkyl, C₁-C₆ haloalkyl, C₁-C₆ alkoxy, C₁-C₆ haloalkoxy, cyano, SR¹¹, N(R^{12a})R^{12b}, C2-C6 alkenyl, aryl, Het³;
R¹¹, R^{12a}, R^{12b}, R¹³, R¹⁴, R¹⁵, R^{16a}, R^{16b}, R¹⁷, R¹⁸, R¹⁹, R²⁰, R^{21a}, R^{21b}, R²², R^{23a}, R^{23b} R^{23c}, R^{23d}, R^{23e} R^{23f}, R^{23g}, R^{23h} R²³ⁱ, R^{23j} and R^{23k} represent independently, at each occurrence, hydrogen or C₁₋₆ alkyl;
m represents an integer selected from 0, 1, 2 or 3;
n represents an integer selected from 1, 2 or 3;
and
X represents nitrogen or oxygen atom.

The compounds of Formula I are referred to hereinafter as “the compound of the invention”.

In another embodiment of the invention there is provided compounds of formula I wherein R¹ and R² represent independently, at each occurrence, halogen, C₁-C₃ alkyl, cyano, SR⁷, N(R^{8a})R^{8b}, C₂-C₃ alkynyl, C₁-C₃ haloalkyl or C₁-C₃ alkoxy;

R³ represents hydrogen or C₁-C₃ alkyl;
R⁴ represents —(CH₂)ₘR⁹ or —(CH₂)ₙOR¹⁰
R³ and R⁴ may together represent a ring;
R⁵ represents hydrogen, C₁-C₃ alkyl (which C₁-C₃ alkyl is optionally substituted by one or more groups selected from halogen, C₂-C₃ alkenyl, C₂-C₃ alkynyl, cyano, —OR¹³, —SR¹⁴, —N(R^{16a})R^{16b});
R⁶ represents hydrogen, C₁-C₃ alkyl or C₁-C₃ alkoxy (which C₁-C₃ alkyl and C₁-C₃ alkoxy are optionally substituted by one or more groups selected from halogen, C₂-C₃ alkenyl, C₂-C₃ alkynyl, cyano, —OR¹⁸, —N(R^{21a})(R^{21b});
R⁷ and R⁸ represent independently, at each occurrence, hydrogen or C₁₋₃ alkyl;
R⁹ and R¹⁰ represents aryl, Het (which aryl and Het optionally are substituted by one or more groups selected from halogen, C₁-C₆ alkyl, C₁-C₆ haloalkyl, C₁-C₆ alkoxy, C₁-C₆ haloalkoxy, cyano, SR¹¹, N(R^{12a})R^{12b}, C₂-C₆ alkenyl, aryl, Het²;
R¹¹, R¹², R¹³, R¹⁴, R¹⁶, R¹⁸, R²¹ and R²³ represent independently, at each occurrence, hydrogen or C₁₋₃ alkyl;
m represents 0 or 1;
n represents 1.

In a further embodiment of the invention there is provided compounds of formula I wherein

R¹ and R² represent independently, at each occurrence, halogen, or C₁-C₃ alkyl;
R³, R⁵ and R⁶ represent hydrogen;
R⁴ represents —(CH₂)ₘR⁹ or —(CH₂)ₙOR¹⁰;
R⁹ and R¹⁰ represent aryl, Het (which aryl and Het groups optionally are substituted by one or more groups selected from halogen, C₁-C₆ alkyl, C₁-C₆ haloalkyl, C₁-C₆ alkoxy, C₁-C₆ haloalkoxy);
m represents 0 or 1;
n represents 1.

Another embodiment of the invention relates to compounds selected from the group consisting of:
N-(2,6-dimethylphenyl)-2-(2-ethoxybenzyl)-3-oxoisoindoline-1-carboxamide;
N-(2-chloro-6-methylphenyl)-2-(2-methoxybenzyl)-3-oxoisoindoline-1-carboxamide;
N-(2,6-dichlorophenyl)-6-fluoro-2-(2-methoxybenzyl)-3-oxoisoindoline-1-carboxamide;
2-(2,3-dihydro-1H-inden-1-yl)-N-(2,6-dimethylphenyl)-3-oxoisoindoline-1-carboxamide;
N-(2,6-dimethylphenyl)-2-(2-isopropoxybenzyl)-3-oxoisoindoline-1-carboxamide;
N-(2,6-dimethylphenyl)-6-fluoro-2-(2-methoxybenzyl)-3-oxoisoindoline-1-carboxamide;
N-(2,6-dimethylphenyl)-3-oxo-2-(1,2,3,4-tetrahydronaphthalen-1-yl)isoindoline-1-carboxamide;
N-(2,6-dimethylphenyl)-2-[1-(2-methoxyphenyl)ethyl]-3-oxoisoindoline-1-carboxamide;
N-(2,6-dimethylphenyl)-2-(2-hydroxybenzyl)-3-oxoisoindoline-1-carboxamide;
N-(2,6-dimethylphenyl)-3-oxo-2-[2-(trifluoromethoxy)benzyl]isoindoline-1-carboxamide;
N-(2,6-dimethylphenyl)-2-{2-[(2,6-dimethylpyridin-3-yl)oxy]ethyl}-3-oxoisoindoline-1-carboxamide;
N-(2,6-dimethylphenyl)-2-{2-[4-fluoro-3-(trifluoromethyl)phenoxy]ethyl}-3-oxoisoindoline-1-carboxamide;
N-(2,6-dimethylphenyl)-2-[(2R)-8-methoxy-1,2,3,4-tetrahydronaphthalen-2-yl]-3-oxoisoindoline-1-carboxamide;
N-(2,6-dimethylphenyl)-2-{1-methyl-2-[3-(trifluoromethyl)phenoxy]ethyl}-3-oxoisoindoline-1-carboxamide;
N-(2,6-dimethylphenyl)-2-{2-[2-fluoro-5-(trifluoromethyl)phenoxy]ethyl}-3-oxoisoindoline-1-carboxamide;
N-(2,6-dichlorophenyl)-2-(2-methoxybenzyl)-3-oxoisoindoline-1-carboxamide;
2-(2,3-dihydro-1,4-benzodioxin-2-ylmethyl)-N-(2,6-dimethylphenyl)-3-oxoisoindoline-1-carboxamide;
N-(2,6-dimethylphenyl)-2-[(2S)-8-methoxy-1,2,3,4-tetrahydronaphthalen-2-yl]-3-oxoisoindoline-1-carboxamide;
N-(2,6-dimethylphenyl)-2-[(3S)-5-methoxy-3,4-dihydro-2H-chromen-3-yl]-3-oxoisoindoline-1-carboxamide;
N-(2,6-dimethylphenyl)-3-oxo-2-[(1R)-1-phenylethyl]isoindoline-1-carboxamide;
N-(2,6-dimethylphenyl)-2-(2-methoxybenzyl)-3-oxoisoindoline-1-carboxamide;
N-(2,6-dimethylphenyl)-2-(2-methylbenzyl)-3-oxoisoindoline-1-carboxamide;
2-[2-(4-chlorophenyl)propyl]-N-(2,6-dimethylphenyl)-3-oxoisoindoline-1-carboxamide;
2-(biphenyl-2-ylmethyl)-N-(2,6-dimethylphenyl)-3-oxoisoindoline-1-carboxamide;
N-(2-isopropyl-6-methylphenyl)-2-(2-methoxybenzyl)-3-oxoisoindoline-1-carboxamide;
2-(2,4-difluorobenzyl)-N-(2-isopropyl-6-methylphenyl)-3-oxoisoindoline-1-carboxamide;
N-(2-isopropyl-6-methylphenyl)-2-[(3-methylpyridin-2-yl)methyl]-3-oxoisoindoline-1-carboxamide;
2-(2-fluorobenzyl)-N-mesityl-3-oxoisoindoline-1-carboxamide;
N-(2,6-dimethylphenyl)-3-oxo-2-(pyridin-2-ylmethyl)isoindoline-1-carboxamide;
2-(3-chlorobenzyl)-N-(2-isopropyl-6-methylphenyl)-3-oxoisoindoline-1-carboxamide;
2-(3-chlorobenzyl)-N-mesityl-3-oxoisoindoline-1-carboxamide;
N-(2,6-dimethylphenyl)-2-[2-(7-methyl-1H-indol-3-yl)ethyl]-3-oxoisoindoline-1-carboxamide;
2-(2,5-dimethoxybenzyl)-N-(2,6-dimethylphenyl)-3-oxoisoindoline-1-carboxamide;
N-(2-chloro-6-methylphenyl)-2-[2-(3-methoxyphenyl)ethyl]-3-oxoisoindoline-1-carboxamide;
N-(2,6-dimethylphenyl)-2-(2-methoxybenzyl)-N-methyl-3-oxoisoindoline-1-carboxamide; and
N-(2,6-dimethoxyphenyl)-2-(2-methoxybenzyl)-3-oxoisoindoline-1-carboxamide.

Unless otherwise specified, “alkyl” groups and “alkoxy” groups as defined herein may be straight-chain or, when there is a sufficient number (i.e. a minimum of three) of carbon atoms be branched-chain, and/or cyclic. Further, when there is a sufficient number (i.e. a minimum of four) of carbon atoms, such alkyl and alkoxy groups may also be part cyclic/acyclic. Unless otherwise specified, alkyl and alkoxy groups may also be substituted by one or more halogen atoms, and especially fluoro atoms. The terms “haloalkyl” and “haloalkoxy” refer to such structures.

“Alkylene” groups as defined herein are divalent and may be straight-chain or, when there is a sufficient number (i.e. a minimum of three) of carbon atoms, be branched-chain. Unless otherwise specified, alkylene groups may also be substituted by one or more halogen atoms, and especially fluoro atoms.

The term “aryl”, when used herein, includes C₆₋₁₀ aryl groups such as phenyl, naphthyl and the like. The term “aryloxy”, when used herein includes C₆₋₁₀ aryloxy groups such as phenoxy, naphthoxy and the like. For the avoidance of doubt, aryloxy groups referred to herein are attached to the rest of the molecule via the O-atom of the oxy-group. Unless otherwise specified, aryl and aryloxy groups may be substituted by one or more substituents including —OH, halo, cyano, nitro, C₁₋₆ alkyl, C₁₋₆ alkoxy or sulfamoyl. When substituted, aryl and aryloxy groups are preferably substituted by between one and three substitutents.

The term “halo”, when used herein, includes fluoro, chloro, bromo and iodo.

Het (Het¹-Het⁵) groups that may be mentioned include those containing 1 to 4 heteroatoms (selected from the group oxygen, nitrogen and/or sulfur) and in which the total number of atoms in the ring system are between five and twelve. Het groups may be fully saturated, wholly aromatic, partly aromatic and/or bicyclic in character. Heterocyclic groups that may be mentioned include benzodioxanyl, benzodioxepanyl, benzodioxinyl, benzodioxolyl, benzofuranyl, benzimidazolyl, benzomorpholinyl, benzoxazinonyl, benzothiophenyl, chromanyl, chromenyl, cinnolinyl, dioxanyl, dioxothiolanyl, furanyl, imidazolyl, imidazo[1,2-a]pyridinyl, indolyl, isoquinolinyl, isoxazolyl, morpholinyl, oxazolyl, phthalazinyl, piperazinyl, piperidinyl, purinyl, pyranyl, pyrazinyl, pyrazolyl, pyridinyl, pyrimindinyl, pyrrolidinonyl, pyrrolidinyl, pyrrolinyl, pyrrolyl, quinazolinyl, quinolinyl, tetrahydropyranyl, tetrahydrofuranyl, thiazolyl, thienyl, thiochromanyl, triazolyl and the like. Substituents on Het groups may, where appropriate, be located on any atom in the ring system including a heteroatom. The point of attachment of Het groups may be via any atom in the ring system including (where appropriate) a heteroatom, or an atom on any fused carbocyclic ring that may be present as part of the ring system. Het groups may also be in the N- or S-oxidised form.

Unless otherwise specified, the Het group may be substituted by one or more substituents including —OH, halo, cyano, nitro, C₁₋₆ alkyl, C₁₋₆ alkoxy or sulfamoyl. When substituted, the Het group is preferably substituted by between one and three substitutents.

Further, the term “hydrocarbon” refers to any structure comprising only carbon and hydrogen atoms.

The term “hydrocarbon radical” or “hydrocarbyl” refers to any structure as a result of removing one or more hydrogens from a hydrocarbon.

The term “alkenyl” refers to a monovalent straight or branched chain alkyl group having at least one carbon-carbon double bond. The double bond of an alkenyl can be unconjugated or conjugated to another unsaturated group. Unless otherwise specified, alkenyl groups as defined herein may be straight-chain or, when there is a sufficient number (i.e. a minimum of three) of carbon atoms be branched-chain, and/or cyclic. Further, when there are a sufficient number (i.e. a minimum of four) of carbon atoms, such alkenyl group may also be part cyclic/acyclic. Unless otherwise specified, alkenyl groups may also be substituted by one or more halogen atoms, and especially fluoro atoms.

The term “alkynyl” refers to a monovalent straight or branched chain alkyl group having at least one carbon-carbon triple bond. The triple bond of an alkynyl can be unconjugated or conjugated to another unsaturated group. Unless otherwise specified, alkynyl groups as defined herein may be straight-chain or, when there is a sufficient number (i.e. a minimum of three) of carbon atoms be branched-chain. Unless otherwise specified, alkenyl groups may also be substituted by one or more halogen atoms, and especially fluoro atoms.

The term “heteroalkyl” refers to a radical formed as a result of replacing one or more carbon atom of an alkyl with one or more heteroatoms selected from N, O and S.

The substituents R³ and R⁴ may together represent a ring. The ring formed by carbon atoms may be 3 up to 8 membered ring, optionally interrupted by O, N or SO₂. The ring may be fused with aryl or Het. Unless otherwise specified the aryl or Het may optionally be substituted by one or more groups selected from halogen, C₁-C₆ alkyl, C₁-C₆ haloalkyl, C₁-C₆ alkoxy, C₁-C₆ haloalkoxy, cyano, phenyl or a further Het. Structures formed by R3 together with R4 that may be mentioned include tetrahydronaphtalene, chromanyl, benzodioxanyl, benzodioxinyl, tetrahydropyranyl, tetrahydrofuranyl.

Pharmaceutically acceptable derivatives include salts and solvates. Salts which may be mentioned include acid addition salts. Specific salts that may be mentioned include arylsulfonate salts, such as toluenesulfonate and, especially, benzenesulfonate salts. Solvates that may be mentioned include hydrates, such as monohydrates of the compounds of the invention.

Pharmaceutically acceptable derivatives also include C₁₋₄ alkyl quaternary ammonium salts and N-oxides.

The compounds of the invention may exhibit tautomerism. All tautomeric forms and mixtures thereof are included within the scope of the invention.

The compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric esters by conventional means (e.g. HPLC, chromatography over silica). All stereoisomers are included within the scope of the invention.

For the avoidance of doubt it is to be understood that where in this specification a group is qualified by ‘hereinbefore defined’, ‘defined hereinbefore’ or ‘defined above’ the said group encompasses the first occurring and broadest definition as well as each and all of the particular definitions for that group.

Compounds of the present invention have been named with the aid of computer software (ACDLabs 8.0/Name(IUPAC)).

Illustrative examples of any substituent, R¹² group or any part of such groups include, but are not limited to:
C₁-C₆ alkyl: methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl;
C₂-C₆ alkenyl: vinyl, allyl, butenyl, pentenyl, hexenyl, cyclohexenyl, butadienyl, pentadienyl, and hexadienyl;
C₂-C₆ alkynyl: ethynyl, propargyl, butynyl, pentynyl;
C₃-C₆ cycloalkyl: cyclopropyl, cyclobutyl, cyclopentyl, and cyclohexyl;

### Preparation

According to the invention there is also provided a process for the preparation of compounds of formula I which comprises:

reaction of a compound of formula II,

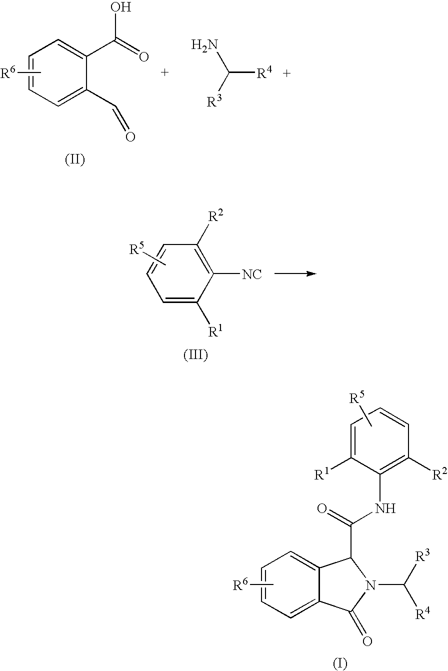

wherein R⁶ is as hereinbefore defined, with an amine R³R⁴CHNH₂ and an phenylisonitrile III under standard Ugi reaction conditions to give compounds of Formula I wherein R¹ to R⁶ are as hereinbefore defined.

According to the invention there is also provided a process for the preparation of compounds of formula I which comprises reaction of a compound of formula IV with an amine V under standard amide coupling reaction conditions.

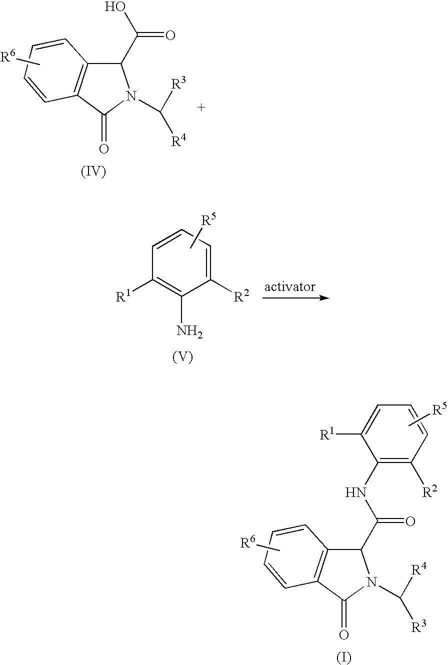

wherein R¹ to R⁶ are as hereinbefore defined.

The skilled person will also appreciate that various standard substituent or functional group interconversions and transformations within certain compounds of formula I will provide other compounds of formula I. For example, carbonyl may be reduced to hydroxy or alkylene, and hydroxy may be converted to halo.

The compounds of the invention may be isolated from their reaction mixtures using conventional techniques.

It will be appreciated by those skilled in the art that, in the process described above, the functional groups of intermediate compounds may be, or may need to be, protected by protecting groups.

Functional groups, which are desirable to protect include hydroxy, amino and carboxylic acid. Suitable protecting groups for hydroxy include trialkylsilyl and diarylalkylsilyl groups (e.g. tert-butyldimethylsilyl, tert-butyldiphenylsilyl or trimethylsilyl), tetrahydropyranyl and alkylcarbonyl groups (e.g. methyl- and ethylcarbonyl groups). Suitable protecting groups for amino include benzyl, sulfonamido (e.g. benzenesulfonamido), tert-butyloxycarbonyl, 9-fluorenyl-methoxycarbonyl or benzyloxycarbonyl. Suitable protecting groups for amidino and guanidino include benzyloxycarbonyl. Suitable protecting groups for carboxylic acid include C₁₋₆ alkyl or benzyl esters.

The protection and deprotection of functional groups may take place before or after any of the reaction steps described hereinbefore.

Protecting groups may be removed in accordance with techniques which are well known to those skilled in the art and as described hereinafter.

The use of protecting groups is fully described in “Protective Groups in Organic Chemistry”, edited by J. W. F. McOmie, Plenum Press (1973), and “Protective Groups in Organic Synthesis”, 3^{rd} edition, T. W. Greene & P. G. M. Wutz, Wiley-Interscience (1999).

Persons skilled in the art will appreciate that, in order to obtain compounds of the invention in an alternative, and, on some occasions, more convenient, manner, the individual process steps mentioned herein may be performed in a different order, and/or the individual reactions may be performed at a different stage in the overall route (i.e. substituents may be added to and/or chemical transformations performed upon, different intermediates to those associated hereinbefore with a particular reaction). This will depend inter alia on factors such as the nature of other functional groups present in a particular substrate, the availability of key intermediates and the protecting group strategy (if any) to be adopted. Clearly, the type of chemistry involved will influence the choice of reagent that is used in the said synthetic steps, the need, and type, of protecting groups that are employed, and the sequence for accomplishing the synthesis.

It will also be appreciated by those skilled in the art that, although certain protected derivatives of compounds of formula I, which may be made prior to a final deprotection stage, may not possess pharmacological activity as such, they may be administered parenterally or orally and thereafter metabolised in the body to form compounds of the invention which are pharmacologically active. Such derivatives may therefore be described as “prodrugs”. Moreover, certain compounds of formula I may act as prodrugs of other compounds of formula I.

All prodrugs of compounds of formula I are included within the scope of the invention.

Some of the intermediates referred to hereinbefore are novel. According to a further aspect of the invention there is thus provided:

### Medical and Pharmaceutical Use

Compounds of the invention are useful because they possess pharmacological activity. They are therefore indicated as pharmaceuticals.

Thus, according to a further aspect of the invention there is provided the compounds of the invention for use as pharmaceuticals.

In particular, the compounds of the invention exhibit voltage-gated sodium channel inhibiting activity, especially NaV1.7 blocking activity, for example as demonstrated in the test described below.

Modulation of voltage-gated sodium channels by pharmacological or genetical tools points to a central role for distinct voltage-gated sodium channels in several disease models. A mouse line has been generated which through advanced molecular biology technologies eliminates the functional expression of NaV1.7 in DRG neurons that express NaV1.8 (Proceedings of the National Academy of Sciences USA (2004) 101(34) 12706-12711). This mouse line shows greatly reduced pain responses in several pain behavior models. Likewise, Herpes-vector mediated knockdown of NaV1.7 in primary afferents of wildtype mice results in a decrease in inflammatory hyperalgesia (Human Gene Therapy (2005) 16(2) 271-277).

Antagonists of NaV channels have been shown to be useful for treating a variety of conditions, including acute, chronic, inflammatory, visceral, and neuropathic pain. More specifically, modulators of NaV activity are currently used or being tested in the clinic as anesthetics, including local anesthetics (Pain (2000) 87(1) 7-17), neuropathic pain reliefers (European Journal of Pain (2002) 6(Supplement 1) 61-68), acute pain reliefers (The Cochrane Database of Systematic Reviews (2005) 3), chronic pain relievers (Pharmacotherapy (2001) 21(9) 1070-1081), inflammatory pain reliefers (Proceedings of the National Academy of Sciences USA (1999) 96(14) 7645-7649), headache reliefers (Headache (2001) 41(Supplement 1) S25-S32).

The compound of the invention are thus expected to be useful in both the prophylaxis and the treatment of a condition which is effected or facilitated by inhibition of voltage-gated sodium channels, in particular acute, chronic, neuropathic, nociceptive, visceral or inflammatory pain

According to a further aspect of the invention, there is provided a method of treatment of any condition mentioned above which method comprises administration of a therapeutically effective amount of a compound of the invention to a person suffering from, or susceptible to, such a condition.

### Pharmaceutical Preparations

The compounds of the invention will normally be administered orally, subcutaneously, intravenously, intraarterially, transdermally, intranasally, by inhalation, or by any other parenteral route, in the form of pharmaceutical preparations comprising the active ingredient either as a free base or a non-toxic organic or inorganic acid addition salt, in a pharmaceutically acceptable dosage form. Depending upon the disorder and patient to be treated, as well as the route of administration, the compositions may be administered at varying doses.

According to a further aspect of the invention there is thus provided a pharmaceutical formulation including a compound of the invention in admixture with a pharmaceutically acceptable adjuvant, diluent or carrier.

Suitable daily doses of the compounds of the invention in therapeutic treatment of humans are about 0.005 to 25.0 mg/kg body weight at oral administration and about 0.005 to 10.0 mg/kg body weight at parenteral administration. Example of ranges of daily doses of the compounds of the invention in therapeutic treatment of humans are about 0.005 to 10.0 mg/kg body weight at oral administration and about 0.005 to 5.0 mg/kg body weight at parenteral administration.

Compounds of the invention may also have the advantage that they may be more efficacious than, be less toxic than, have a broader range of activity than, be more potent than, be longer acting than, produce fewer side effects than, be more easily absorbed than, or that they may have other useful pharmacological properties over, compounds known in the prior art.

The invention is illustrated by way of the following examples.

### EXAMPLES

### General Experimental Procedures

Mass spectra were recorded on one of the following instruments: a Perkin-Elmer SciX API 150ex spectrometer; a VG Quattro II triple quadrupole; a VG Platform II single quadrupole; or a Micromass Platform LCZ single quadrupole mass spectrometer (the latter three instruments were equipped with a pneumatically assisted electrospray interface (LC-MS)). NMR spectra were recorded on a Varian Unity+ 400 NMR Spectrometer, operating at 400 MHz for proton and 100 MHz for carbon-13, and equipped with a 5 mm BBO probe with Z-gradients; or on a Bruker av400 NMR spectrometer operating at 400 MHz for proton and 100 MHz for carbon-13, and equipped with a 3 mm flow injection SEI ¹H/D-¹³C probehead with Z-gradients, using a BEST 215 liquid handler for sample injection; or on a Bruker DPX400 NMR spectrometer, operating at 400 MHz for proton and 100 MHz for carbon-13, and equipped with a 4-nucleus probe with Z-gradients; or on a Bruker DRX600 NMR Spectrometer, operating at 600 MHz for proton and 150 MHz for carbon-13, and equipped with a 5 mm BBO probe with Z-gradients or a Snun TXI probe with Z-gradients or a 2.5 mm BBI probe with Z-gradients. The following reference signals were used: TMS δ 0.00, or the residual solvent signal of DMSO-d₆ δ 2.49, CD₃OD δ 3.30, acetone-d₆ 2.04 or CDCl₃ δ 7.25 (unless otherwise indicated). Resonance multiplicities are denoted s, d, t, q, m, br and app for singlet, doublet, triplet, quartet, multiplet, broad and apparent, respectively.

Rotamers may or may not be denoted in spectra depending upon ease of interpretation of spectra. Unless otherwise stated, chemical shifts are given in ppm with the solvent as internal standard.

### Synthesis of Intermediates

The following intermediates were not commercially available, and were therefore prepared by method A described below:
2-[2-fluoro-5-(trifluoromethyl)phenoxy]ethanamine
2-[4-fluoro-3-(trifluoromethyl)phenoxy]ethanamine
2-[(2,6-dimethylpyridin-3-yl)oxy]ethanamine.

### Preparation A

To an appropriate phenol in dioxane (10 mL) aziridine (3-9 equiv) was added and the resulting mixture was heated at 70-100° C. for several days. The volatiles were removed in vacuum and the residue was purified by column chromatography on silica gel using a gradient of methanol in dichloromethane as an eluent to afford the desired compound.

### Synthesis of Compounds of Formula I

### General Reaction Procedure

Phthalaldehydic acid (0.2-0.5 mmol) and the amine (1 equivalent) were dissolved in methanol. Solution of the isonitrile (1 equiv) in methanol was added. The reaction mixture was stirred at room temperature overnight. The solvent was removed in vacuo and the residue was dissolved in chloroform and washed with water and brine. The organic layer was dried over MgSO₄ and concentrated in vacuo. The crude product was purified either by flash chromatography on silica gel or using preparative HPLC technique on reversed stationary phase.

### Example 1

### N-(2,6-dimethylphenyl)-2-(2-ethoxybenzyl)-3-oxoisoindoline-1-carboxamide

To a solution of phthalaldehydic acid, (60 mg, 0.4 mmol) and 1-(2-ethoxyphenyl)methanamine (60 mg, 0.4 mmol) in methanol (1 ml) a solution of 2,6-dimethylphenyl isocyanide (53 mg, 0.4 mmol) in methanol (1 ml) was added. The reaction mixture was stirred at room temperature overnight. The volatiles were removed in vacuum. The residue dissolved in chloroform was washed with water and brine. The organic layer was dried over MgSO₄ and concentrated in vacuum. The crude product was purified by flash chromatography using a gradient of ethyl acetate in heptane as an eluent yielding the title compound (mg, 74%). ¹H NMR (400 MHz, CDCl₃) δ (ppm) 7.89 (d, 1H), 7.70 (d, 1H), 7.50-7.61 (m, 2H), 7.36 (dd, 1H), 7.27-7.30 (m, 1H), 7.23-7.27 (m, 1H), 7.07-7.12 (m, 1H), 7.01-7.06 (m, 2H), 6.85-6.93 (m, 2H), 5.41 (d, 1H), 5.11 (s, 1H), 4.72 (d, 1H), 4.00-4.15 (m, 2H), 2.02 (s, 6H), 1.39 (t, 3H); MS (ESI) m/z 415 [M+1].

### Example 2-34

The following compounds were prepared, from appropriate intermediates (such as those described hereinbefore), according to or by analogy with methods described herein and/or by standard solid or solution phase parallel chemistry techniques

| | | Mass | |
|---|---|---|---|
| Example | | spectrum | |
| # | Compound name | (ESI) m/z | 1H NMR spectrum |
| 2 | N-(2-chloro-6-methylphenyl)-2-(2- | 421, 423 | (400 MHz, DMSO-d₆) δ |
| | methoxybenzyl)-3-oxoisoindoline-1- | | (ppm) 10.32 (s, 1 H), 7.73-7.79 |
| | carboxamide | | (m, 2 H), 7.67 (dt, 1 H), |
| | | | 7.57 (t, 1 H), 7.34-7.39 (m, |
| | | | 1 H), 7.27-7.33 (m, 1 H), |
| | | | 7.21-7.27 (m, 2 H), 7.18 |
| | | | (dd, 1 H), 7.05 (d, 1 H), 6.93 |
| | | | (dt, 1 H), 5.27 (s, 1 H), 5.10 |
| | | | (d, 1 H), 4.31 (d, 1 H), 3.81 |
| | | | (s, 3 H), 2.13 (s, 3 H); |
| 3 | N-(2,6-dichlorophenyl)-6-fluoro-2-(2- | 459, | (400 MHz, CDCl₃) δ (ppm) |
| | methoxybenzyl)-3-oxoisoindoline-1- | 461, 463 | 8.13 (s, 1 H), 7.73 (dd, 1 H), |
| | carboxamide | | 7.38 (d, 2 H), 7.27-7.36 (m, |
| | | | 2 H), 7.12-7.26 (m, 3 H), |
| | | | 6.85-6.94 (m, 2 H), 5.36 (d, |
| | | | 1 H), 5.05 (s, 1 H), 4.83 (d, 1 |
| | | | H), 3.83 (s, 3 H) |
| 4 | 2-(2,3-dihydro-1H-inden-1-yl)-N-(2,6- | 397 | (600 MHz, DMSO-d₆) δ |
| | dimethylphenyl)-3-oxoisoindoline-1- | | (ppm) 9.93 (s, 1 H), 7.79 (d, |
| | carboxamide | | 1 H), 7.70-7.74 (m, 1 H), |
| | | | 7.67 (t, 1 H), 7.31-7.35 (m, |
| | | | 1 H), 7.27 (t, 1 H), 7.14-7.21 |
| | | | (m, 2 H), 6.98-7.08 |
| | | | (m, 4 H), 5.79 (t, 1 H), 5.31 |
| | | | (s, 1 H), 2.95-3.03 (m, 1 |
| | | | H), 2.84-2.93 (m, 1 H), |
| | | | 2.39-2.47 (m, 1 H), 2.26-2.37 |
| | | | (m, 1 H), 1.95 (s, 6 H) |
| 5 | N-(2,6-dimethylphenyl)-2-(2- | 429 | (400 MHz, DMSO-d₆) δ |
| | isopropoxybenzyl)-3-oxoisoindoline- | | ppm 9.89 (s, 1 H), 7.79 (d, 1 |
| | 1-carboxamide | | H), 7.64-7.74 (m, 2 H), |
| | | | 7.58 (t, 1 H), 7.24-7.31 (m, |
| | | | 1 H), 7.14 (dd, 1 H), 7.00- |
| | | | 7.11 (m, 4 H), 6.90 (t, 1 H), |
| | | | 5.18 (d, 1 H), 5.13 (s, 1 H), |
| | | | 4.58-4.68 (m, 1 H), 4.21 (d, |
| | | | 1 H), 2.07 (s, 6 H), 1.24 (d, 3 |
| | | | H), 1.10 (d, 3 H) |
| 6 | N-(2,6-dimethylphenyl)-6-fluoro-2- | 419 | (400 MHz, CDCl₃) δ (ppm) |
| | (2-methoxybenzyl)-3-oxoisoindoline- | | 7.87 (dd, 1 H), 7.28-7.43 |
| | 1-carboxamide | | (m, 3 H), 7.18-7.26 (m, 2 |
| | | | H), 7.09-7.15 (m, 1 H), |
| | | | 7.03-7.09 (m, 2 H), 6.88-6.97 |
| | | | (m, 2 H), 5.37 (d, 1 H), |
| | | | 5.02 (s, 1 H), 4.72 (d, 1 H), |
| | | | 3.85 (s, 3 H), 2.06 (s, 6 H); |
| 7 | N-(2,6-dimethylphenyl)-3-oxo-2- | 411 | ¹H NMR (600 MHz, CDCl₃) |
| | (1,2,3,4-tetrahydronaphthalen-1- | | δ (ppm) 7.98-8.06 (m, 1 H), |
| | yl)isoindoline-1-carboxamide | | 7.57-7.69 (m, 3 H), 7.10-7.18 |
| | | | (m, 3 H), 7.04-7.10 |
| | | | (m, 1 H), 6.93-7.03 (m, 3 |
| | | | H), 6.66 (d, 1 H), 5.94 (dd, 1 |
| | | | H), 4.97 (s, 1 H), 2.89-2.98 |
| | | | (m, 1 H), 2.79-2.89 (m, 1 |
| | | | H), 2.41-2.50 (m, 1 H), |
| | | | 2.17-2.24 (m, 1 H), 2.09-2.17 |
| | | | (m, 1 H), 1.91-2.02 |
| | | | (m, 1 H), 1.87 (s, 6 H) |
| 8 | N-(2,6-dimethylphenyl)-2-[1-(2- | 415 | (400 MHz, CDCl₃) δ (ppm) |
| | methoxyphenyl)ethyl]-3- | | 7.86-7.91 (m, 1 H), 7.68 (d, |
| | oxoisoindoline-1-carboxamide | | 1 H), 7.63 (dd, 1 H), 7.49-7.59 |
| | | | (m, 2 H), 7.38 (s, 1 H), |
| | | | 7.28-7.32 (m, 1 H), 6.99-7.08 |
| | | | (m, 2 H), 6.91-6.98 |
| | | | (m, 2 H), 6.80 (d, 1 H), 5.82 |
| | | | (q, 1 H), 5.24 (s, 1 H), 3.78 |
| | | | (s, 3 H), 1.90 (d, 3 H), 1.72 |
| | | | (s, 6 H); |
| 9 | N-(2,6-dimethylphenyl)-2-(2- | 387 | (400 MHz, DMSO-d₆) δ |
| | hydroxybenzyl)-3-oxoisoindoline-1- | | ppm 9.96 (s, 1 H) 9.67 (s, 1 |
| | carboxamide | | H) 7.71-7.79 (m, 2 H) 7.67 |
| | | | (dt, 1 H) 7.57 (t, 1 H) 7.04- |
| | | | 7.16 (m, 5 H) 6.87 (d, 1 H) |
| | | | 6.77 (dt, 1 H) 5.23 (s, 1 H) |
| | | | 5.10 (d, 1 H) 4.26 (d, 1 H) |
| | | | 2.09 (s, 6 H). |
| 10 | N-(2,6-dimethylphenyl)-3-oxo-2-[2- | 455 | (400 MHz, DMSO-d₆) δ |
| | (trifluoromethoxy)benzyl]isoindoline- | | ppm 10.03 (s, 1 H) 7.82 (d, 1 |
| | 1-carboxamide | | H) 7.68-7.77 (m, 2 H) 7.60 |
| | | | (t, 1 H) 7.38-7.53 (m, 4 H) |
| | | | 7.04-7.13 (m, 3 H) 5.31 (d, |
| | | | 1 H) 5.23 (s, 1 H) 4.28 (d, 1 |
| | | | H) 2.07 (s, 6 H) |
| 11 | N-(2,6-dimethylphenyl)-2-{2-[(2,6- | 430 | (400 MHz, DMSO-d₆) δ |
| | dimethylpyridin-3-yl)oxy]ethyl}-3- | | ppm 10.02 (s, 1 H) 7.74-7.84 |
| | oxoisoindoline-1-carboxamide | | (m, 2 H) 7.70 (dt, 1 H) |
| | | | 7.58 (t, 1 H) 7.28 (d, 1 H) |
| | | | 7.04-7.13 (m, 3 H) 7.00 (d, |
| | | | 1 H) 5.61 (s, 1 H) 4.33-4.43 |
| | | | (m, 1 H) 4.20-4.32 (m, 2 H) |
| | | | 3.38-3.48 (m, 1 H) 2.33 (s, |
| | | | 3 H) 2.29 (s, 3 H) 2.08 (s, 6 |
| | | | H) |
| 12 | N-(2,6-dimethylphenyl)-2-{2-[4- | 487 | (400 MHz, DMSO-d₆) δ |
| | fluoro-3- | | ppm 10.11 (s, 1 H) 7.78 (t, 2 |
| | (trifluoromethyl)phenoxy]ethyl}-3- | | H) 7.71 (dt, 1 H) 7.58 (t, 1 |
| | oxoisoindoline-1-carboxamide | | H) 7.47 (t, 1 H) 7.34-7.42 |
| | | | (m, 2 H) 7.06-7.14 (m, 3 H) |
| | | | 5.69 (s, 1 H) 4.26-4.43 (m, |
| | | | 3 H) 3.36-3.45 (m, 1 H) |
| | | | 2.10 (s, 6 H) |
| 13 | N-(2,6-dimethylphenyl)-2-[(2R)-8- | 441 | |
| | methoxy-1,2,3,4- | | |
| | tetrahydronaphthalen-2-yl]-3- | | |
| | oxoisoindoline-1-carboxamide | | |
| 14 | N-(2,6-dimethylphenyl)-2-{1-methyl- | 483 | |
| | 2-[3-(trifluoromethyl)phenoxy]ethyl}- | | |
| | 3-oxoisoindoline-1-carboxamide | | |
| 15 | N-(2,6-dimethylphenyl)-2-{2-[2- | 487 | (400 MHz, DMSO-d₆) δ |
| | fluoro-5- | | ppm 10.02 (s, 1 H) 7.81 (d, 1 |
| | (trifluoromethyl)phenoxy]ethyl}-3- | | H) 7.77 (d, 1 H) 7.70 (dt, 1 |
| | oxoisoindoline-1-carboxamide | | H) 7.55-7.64 (m, 2 H) 7.42-7.50 |
| | | | (m, 1 H) 7.33-7.40 |
| | | | (m, 1 H) 7.04-7.13 (m, 3 H) |
| | | | 5.63 (s, 1 H) 4.45-4.52 (m, |
| | | | 2 H) 4.36-4.44 (m, 1 H) |
| | | | 3.41-3.50 (m, 1 H) 2.08 (s, |
| | | | 6 H) |
| 16 | N-(2,6-dichlorophenyl)-2-(2- | 441 | (400 MHz, DMSO-d₆) δ |
| | methoxybenzyl)-3-oxoisoindoline-1- | | ppm 10.66 (s, 1 H) 7.73-7.81 |
| | carboxamide | | (m, 2 H) 7.64-7.71 (m, |
| | | | 1 H) 7.54-7.62 (m, 3 H) |
| | | | 7.38 (t, 1 H) 7.27-7.34 (m, |
| | | | 1 H) 7.18 (dd, 1 H) 7.04 (d, |
| | | | 1 H) 6.93 (t, 1 H) 5.28 (s, 1 |
| | | | H) 5.10 (d, 1 H) 4.32 (d, 1 |
| | | | H) 3.80 (s, 3 H) |
| 17 | 2-(2,3-dihydro-1,4-benzodioxin-2- | 429 | (400 MHz, DMSO-d₆) δ |
| | ylmethyl)-N-(2,6-dimethylphenyl)-3- | | ppm (mixture of |
| | oxoisoindoline-1-carboxamide | | diastereomers 1:1) 10.19 (s, |
| | | | 1 H) 10.07 (s, 1 H) 7.67-7.87 |
| | | | (m, 6 H) 7.55-7.63 (m, |
| | | | 2 H) 6.79-7.15 (m, 14 H) |
| | | | 5.77 (s, 1 H) 5.68 (s, 1 H) |
| | | | 4.47-4.59 (m, 2 H) 4.34-4.44 |
| | | | (m, 2 H) 4.25-4.35 (m, |
| | | | 2 H) 4.12 (dd, 1 H) 3.95 (dd, |
| | | | 1 H) 3.44 (dd, 1 H) 3.21 (dd, |
| | | | 1 H) 2.16 (s, 6 H) 2.00 (s, 6 |
| | | | H) |
| 18 | N-(2,6-dimethylphenyl)-2-[(2S)-8- | 441 | |
| | methoxy-1,2,3,4- | | |
| | tetrahydronaphthalen-2-yl]-3- | | |
| | oxoisoindoline-1-carboxamide | | |
| 19 | N-(2,6-dimethylphenyl)-2-[(3S)-5- | 443 | |
| | methoxy-3,4-dihydro-2H-chromen-3- | | |
| | yl]-3-oxoisoindoline-1-carboxamide | | |
| 20 | N-(2,6-dimethylphenyl)-3-oxo-2- | 385 | (400 MHz, DMSO-d₆) δ |
| | [(1R)-1-phenylethyl]isoindoline-1- | | ppm (only signals |
| | carboxamide | | corresponding to the major |
| | | | isomer included)10.04 (s, 1 |
| | | | H) 7.69-7.77 (m, 2 H) 7.63-7.69 |
| | | | (m, 1 H) 7.56 (t, 1 H) |
| | | | 7.35-7.41 (m, 2 H) 7.28-7.34 |
| | | | (m, 3 H) 7.02-7.10 (m, |
| | | | 3 H) 5.60 (q, 1 H) 5.30 (s, 1 |
| | | | H) 2.08 (s, 6 H) 1.67 (d, 3 |
| | | | H) |
| 21 | N-(2,6-dimethylphenyl)-2-(2- | 401 | (400 MHz, DMSO-d₆) δ |
| | methoxybenzyl)-3-oxoisoindoline-1- | | ppm 10.00 (s, 1 H) 7.77 (d, 1 |
| | carboxamide | | H) 7.65-7.75 (m, 2 H) 7.58 |
| | | | (t, 1 H) 7.28-7.35 (m, 1 H) |
| | | | 7.18 (dd, 1 H) 7.03-7.14 |
| | | | (m, 4 H) 6.94 (t, 1 H) 5.24 |
| | | | (s, 1 H) 5.09 (d, 1 H) 4.29 |
| | | | (d, 1 H) 3.81 (s, 3 H) 2.10 (s, |
| | | | 6 H) |
| 22 | N-(2,6-dimethylphenyl)-2-(2- | | |
| | methylbenzyl)-3-oxoisoindoline-1- | | |
| | carboxamide | | |
| 23 | 2-[2-(4-chlorophenyl)propyl]-N-(2,6- | | |
| | dimethylphenyl)-3-oxoisoindoline-1- | | |
| | carboxamide | | |
| 24 | 2-(biphenyl-2-ylmethyl)-N-(2,6- | | |
| | dimethylphenyl)-3-oxoisoindoline-1- | | |
| | carboxamide | | |
| 25 | N-(2-isopropyl-6-methylphenyl)-2-(2- | 429 | |
| | methoxybenzyl)-3-oxoisoindoline-1- | | |
| | carboxamide | | |
| 26 | 2-(2,4-difluorobenzyl)-N-(2- | 435 | |
| | isopropyl-6-methylphenyl)-3- | | |
| | oxoisoindoline-1-carboxamide | | |
| 27 | N-(2-isopropyl-6-methylphenyl)-2- | 414 | |
| | [(3-methylpyridin-2-yl)methyl]-3- | | |
| | oxoisoindoline-1-carboxamide | | |
| 28 | 2-(2-fluorobenzyl)-N-mesityl-3- | 403 | |
| | oxoisoindoline-1-carboxamide | | |
| 29 | N-(2,6-dimethylphenyl)-3-oxo-2- | 372 | |
| | (pyridin-2-ylmethyl)isoindoline-1- | | |
| | carboxamide | | |
| 30 | 2-(3-chlorobenzyl)-N-(2-isopropyl-6- | 433 | |
| | methylphenyl)-3-oxoisoindoline-1- | | |
| | carboxamide | | |
| 31 | 2-(3-chlorobenzyl)-N-mesityl-3- | | |
| | oxoisoindoline-1-carboxamide | | |
| 32 | N-(2,6-dimethylphenyl)-2-[2-(7- | | |
| | methyl-1H-indol-3-yl)ethyl]-3- | | |
| | oxoisoindoline-1-carboxamide | | |
| 33 | 2-(2,5-dimethoxybenzyl)-N-(2,6- | | |
| | dimethylphenyl)-3-oxoisoindoline-1- | | |
| | carboxamide | | |
| 34 | N-(2-chloro-6-methylphenyl)-2-[2-(3- | | |
| | methoxyphenyl)ethyl]-3- | | |
| | oxoisoindoline-1-carboxamide | | |

### Example 35

### N-(2,6-dimethylphenyl)-2-(2-methoxybenzyl)-N-methyl-3-oxoisoindoline-1-carboxamide

To a solution of N-(2,6-dimethylphenyl)-2-(2-methoxybenzyl)-3-oxoisoindoline-1-carboxamide (96 mg, 0.24 mmol) in THF (6 mL) butyllithium (2.5M in hexanes, 106 μL, 0.26 mmol) was added at −45° C. under argon. The reaction mixture was stirred at −45° C. for 15 minutes before methyl triflate (80 μL, 0.71 mmol) was added. After 30 minutes the reaction mixture was quenched by the addition of water (20 mL) followed by extraction with dichloromethane (3×20 mL). The organic phase was dried over magnesium sulphate and concentrated in vacuum. The crude product was purified by preparative HPLC to afford the target compound (14 mg, 14%).

1H NMR (400 MHz, CHLOROFORM-d) δ ppm (mixture of rotamers 1:1) 7.94 (d, 1H) 7.79-7.85 (m, 1H) 7.51-7.64 (m, 3H) 7.39-7.47 (m, 3H) 7.25-7.32 (m, 1H) 7.20-7.25 (m, 2H) 7.06-7.20 (m, 5H) 6.82-6.98 (m, 5H) 6.76-6.83 (m, 1H) 5.58 (br. s., 1H) 5.38 (d, 1H) 5.21 (d, 1H) 5.12 (s, 1H) 4.45-4.59 (m, 1H) 4.38 (d, 1H) 3.86 (s, 3H) 3.83 (s, 3H) 3.22 (s, 3H) 3.08 (br. s., 3H) 2.42 (s, 3H) 2.26 (br. s., 3H) 2.16 (br. s., 3H) 1.82 (s, 3H)

MS (ES) m/z 415 (M+1).

### Example 36

### N-(2,6-dimethoxyphenyl)-2-(2-methoxybenzyl)-3-oxoisoindoline-1-carboxamide

### A. Ethyl 2-(2-methoxybenzyl-3-oxoisoindoline-1-carboxylate

To a solution of ethyl 2-(1-bromo-2-ethoxy-2-oxoethyl)benzoate (1.58 g, 5 mmol) in dry acetonitrile 2-methoxybenzylamine (1.3 mL, 10 mmol) was added at 0-5° C. under argon atmosphere. The reaction mixture was stirred at room temperature for 12 h. The precipitate was filtered off, and washed with EtOAc (50 mL). The combined solutions were concentrated in vacuum. The residue was purified by column chromatography at silica gel, using heptane in ethyl acetate (3:1) as an eluent to yield ethyl 2-(2-methoxybenzyl)-3-oxoisoindoline-1-carboxylate as yellow oil (1.46 g, 90%).

¹H NMR (400 MHz, DMSO-d₆) δ ppm 1.23 (t, 3H) 3.81 (s, 3H) 4.15-4.27 (m, 2H) 4.41 (d, 1H) 5.01 (d, 1H) 5.14 (s, 1H) 6.89-6.95 (m, 1H) 7.04 (d, 1H) 7.17 (dd, 1H) 7.28-7.34 (m, 1H) 7.55-7.62 (m, 2H) 7.63-7.68 (m, 1H) 7.77 (d, 1H) [M+H] 326, [M−H] 324.

### B. 2-(2-Methoxybenzyl-3-oxoisoindoline-1-carboxylic acid

To a solution of ethyl 2-(2-methoxybenzyl)-3-oxoisoindoline-1-carboxylate (1.46 g, 4.5 mmol) in methanol (15 mL) sodium hydroxide (13.5 mL of 1 M aqueous solution) was added. The reaction mixture was heated at 35° C. for 40 min. The mixture was cooled down to room temperature, and treated with 2 M hydrochloric acid. The methanol was removed in vacuum, and the residue was extracted with ethyl acetate (3×75 mL). The organic phase was dried over MgSO₄, and concentrated in vacuum to yield 2-(2-methoxybenzyl)-3-oxoisoindoline-1-carboxylic acid as yellow solid (1.29 g, 96%).

¹H NMR (400 MHz, DMSO-d₆) δ ppm 3.81 (s, 3H) 4.39 (d, 2H) 5.00-5.07 (m, 2H) 6.92 (t, 1H) 7.04 (d, 1H) 7.15 (d, 1H) 7.27-7.34 (m, 1H) 7.54-7.60 (m, 1H) 7.61-7.69 (m, 2H) 7.75 (d, 1H) [M+H] 298, [M−H] 296.

### C. N-(2,6-Dimethoxyphenyl)-2-(2-methoxybenzyl)-3-oxoisoindoline-1-carboxamide

To a solution of 2-(2-methoxybenzyl)-3-oxoisoindoline-1-carboxylic acid (59 mg, 0.2 mmol), and NEt₃ (84 μL, 0.3 mmol) in DMF (2 mL), N,N,N′,N′-tetramethylfluoroformamidinium hexafluorophosphate (79 mg, 0.3 mmol) was added, and the mixture was stirred at room temperature for 30 min. 2,6-Dimethoxyaniline (46 mg, 0.3 mmol) was added in one portion, and the reaction mixture was heated at 50° C. for 45 min. The mixture was cooled down to room temperature, filtered, and purified by preparative HPLC to yield the title compound (15 mg, 17%).

### Example 37

### N-(2,6-dimethylphenyl)-2-(4-fluoro-2-methoxybenzyl)-5-hydroxy-4-methyl-3-oxoisoindoline-1-carboxamide

To a solution of 2-furaldehyde (41 μL, 0.5 mmol) in methanol (1.5 mL) 4-fluoro-2-methoxybenzylamine (86 mg, 0.55 mmol), 2,6-dimethylphenyl isocyanide (72 mg, 0.55 mmol) and 2-butynoic acid (46 mg, 0.55 mmol) were added sequentially. The reaction mixture was stirred overnight. The volatiles were removed in vacuum and the residue was dissolved in dioxane (3 mL). Ytterbium trifluoromethanesulphonate (62 mg, 0.1 mmol) was added and the solution was heated at 100° C. by microwave irradiation for 15 min. The mixture was cooled down to ambient temperature, filtered, and purified using preparative HPLC. The fractions containing the target compound were combined and extracted with ethyl acetate. The organic layer was dried with magnesium sulfate, and concentrated in vacuum to yielded the titled compound as yellowish solid (74 mg, 33%).

1H NMR (400 MHz, CDCl₃) δ ppm 7.43 (s, 1H), 7.18-7.23 (m, 1H), 7.02-7.11 (m, 3H), 6.53-6.61 (m, 2H), 6.45-6.50 (m, 1H), 6.23-6.26 (m, 1H), 5.95 (s, 1H), 4.85-4.98 (m, 2H), 3.75 (s, 3H), 2.17 (s, 6H), 1.96 (s, 3H)

MS (ESI) m/z 449 [M+H], MS (ESI) m/z 447 [M−H]

### Example 38

### N-(2,6-dimethylphenyl)-2-(4-fluoro-2-methoxybenzyl)-3-oxoisoindoline-1-carboxamide

### Step A

### Ethyl 2-(4-fluoro-2-methoxybenzyl)-3-oxoisoindoline-1-carboxylate

To a stirred solution of ethyl 2-(1-bromo-2-ethoxy-2-oxoethyl)benzoate (Othman, M.; Decroix, B: Synth. Comm. 1996, 26, 2803) (945 mg, 3 mmol) in acetonitrile (15 mL) containing triethyl amine (835 μL, 6 mmol) a solution of 4-fluoro-2-methoxybenzylamine (698 mg, 4.5 mmol) in acetonitrile (1 mL) was added dropwise under argon at 0° C. The reaction mixture was stirred at ambient temperature for 2 h. The mixture was diluted with ethyl acetate (50 mL), washed with water and brine. The organic phase was dried over magnesium sulfate and concentrated in vacuum. The residue was purified by column chromatography on silica gel using heptane/ethyl acetate (3:2) as an eluent to yield ethyl 2-(4-fluoro-2-methoxybenzyl)-3-oxoisoindoline-1-carboxylate as yellowish oil (857 mg, 83%).

1H NMR (400 MHz, CDCl₃) δ ppm 7.84-7.88 (m, 1H), 7.67-7.76 (m, 1H), 7.59-7.65 (m, 1H), 7.46-7.58 (m, 2H), 7.28-7.33 (m, 1H), 6.58-6.66 (m, 1H), 5.21 (d, 1H), 4.96 (s, 1H), 4.47 (d, 1H), 4.17-4.38 (m, 2H), 3.82 (s, 3H), 1.32 (q, 3H)

MS (ESI) m/z 344 [M+H], MS (ESI) m/z 342 [M−H]

### Step B

### 2-(4-Fluoro-2-methoxybenzyl)-3-oxoisoindoline-1-carboxylic acid

To a stirred solution of ethyl 2-(4-fluoro-2-methoxybenzyl)-3-oxoisoindoline-1-carboxylate (443 mg, 1.3 mmol) in methanol (10 mL) sodium hydroxide (1M, 4 mL) was added. The mixture was stirred at ambient temperature for 30 minutes, then treated with hydrochloric acid (2M) to reach pH 2. The methanol was evaporated in vacuum and the residual water phase was extracted with ethyl acetate. The organic phase was dried over magnesium sulfate and concentrated to yield 2-(4-fluoro-2-methoxybenzyl)-3-oxoisoindoline-1-carboxylic acid as yellow oil (756 mg, 96%).

1H NMR (400 MHz, MeOD) δ ppm 7.76-7.80 (m, 1H), 7.56-7.61 (m, 1H), 7.46-7.54 (m, 2H), 7.20-7.25 (m, 1H), 6.79-6.84 (m, 1H), 6.63-6.69 (m, 1H), 4.78 (s, 1H), 4.38 (s, 2H), 3.86 (s, 3H)

MS (ESI) m/z 316 [M+H]

### Step C

To a solution of 2-(4-fluoro-2-methoxybenzyl)-3-oxoisoindoline-1-carboxylic acid (63 mg, 0.2 mmol) in dimethylformamide (1 mL) triethyl amine (84 μL, 0.4 mmol) was added followed by N,N,N′,N′-tetramethylfluoroformamidiniumhexafluuorophosphate (79 mg, 0.3 mmol). The mixture was stirred at ambient temperature for 5 minutes. To the reaction mixture 2,6-dimethylaniline (75 μL, 0.6 mmol) was added dropwise, and the mixture was heated at 45° C. for 1 h. The mixture was filtered and purified by HPLC. The fractions containing the target compound were collected and extracted with ethyl acetate. The organic phase was dried with magnesium sulfate and concentrated ion vacuum to yield N-(2,6-dimethylphenyl)-2-(4-fluoro-2-methoxybenzyl)-3-oxoisoindoline-1-carboxamide as white solid (8.8 mg, 10%).

1H NMR (400 MHz, CDCl₃) δ ppm 7.89 (d, 1H), 7.67-7.72 (m, 1H), 7.57-7.62 (m, 1H), 7.52-7.57 (m, 1H), 7.31-7.37 (m, 1H), 7.18 (s, 1H), 7.08-7.14 (m, 1H), 7.03-7.07 (m, 2H), 6.64 (d, 2H), 5.36 (d, 1H), 5.02 (s, 1H), 4.65 (d, 1H), 3.85 (s, 3H), 2.03 (s, 6H)

MS (ESI) m/z 419 [M+H], MS (ESI) m/z 417 [M−H]

### Biological Tests

### Expression of Voltage-Gated Sodium Channel in Cell Lines:

Gene(s) encoding the full-length protein of the voltage-gated sodium channel of interest are cloned and expressed under a suitable promoter in a suitable cell line, as well known in the art. The so constructed stable cell lines are used in screening assays to identify suitable compounds active on voltage-gated sodium channels. Suitable screening assays are as follows.

### Li⁺ Influx Assay

The cell line expressing the voltage-gated sodium channel of interest was plated in conventional 96 or 384 well tissue plates at a suitable cell density (for example 40000 cells/well in 96 well plate, or 20000 cells/well in 384 well plate). The cells were then repeatedly washed with a suitable Na free buffer using a suitable commercially available washer (for example EL-405 washer) until all tissue culture medium was removed from the wells. A suitable Na-free buffer could have the composition (mM) Choline chloride 137, KCl 5.4, MgSO4 0.81, CaCl2 0.95, glucose 5.55 and HEPES 25 at pH 7.4, but may also have other suitable composition. After completion of all wash steps, cells were incubated in the suitable Na free buffer for 15 minutes. Then, the Na free buffer was removed and cells were incubated with a buffer rich in LiCl for 60 minutes at 37° C. The LiCl buffer was also enriched in potassium ions, causing a depolarizing stimulus to the cells. Such a buffer may have the composition (mM): LiCl 100, KCl 50, MgSO4 0.81, CaCl2 0.95, glucose 5.55 and HEPES 25 at pH 7.4, but may also have other suitable composition. To enhance signal-to-noise ratio, an effective concentration (for example 100 μM) of the voltage-gated sodium channel opener veratridine, or any other suitable voltage-gated sodium channel opener, may be added to the medium to enhance signal detection. Furthermore, and also to enhance signal-to-noise ratio, an effective concentration (for example 10 μg/ml) of suitable scorpion venom may also be added to the medium to delay channel inactivation. In order to find a modulator of the voltage-gated sodium channel of interest, the assay could be complemented with compounds from a compound library.

Compounds of interest were added to the Li-rich solution, one in each well. At the end of the incubation period cells were repeatedly washed with Na free buffer until all extracellular LiCl was removed. Cell lysis was obtained through incubation of cells with triton (1%) for 15 min, or any other suitable method. The resulting cell lysate was then introduced into an atomic absorption spectrophotometer, thus quantifying the amount of Li-influx during the procedure described above.

The described assay can be run with any atomic absorption spectrophotometer using plates of 96-well format, 384-well format, or any other conventional plate format. The described assay can be applied to cell lines expressing any given one or more of the voltage-gated sodium channel alpha subunits, as well as any given combination of one of the voltage-gated alpha subunits with any one or more beta subunit.

If needed the cell line of choice can be further hyperpolarised by expression of a suitable potassium leak ion channel, for example TREK-1, either by transient co-transfection or through establishment of a stable co-transfected cell line. The successful expression of a leak K current can be verified using traditional intracellular electrophysiology, either in whole cell patch-clamp, perforated patch-clamp or conventional two-electrode voltage-clamp. A cell line of choice modified to successfully express a voltage-gated sodium channel of interest together with a suitable potassium leak ion channel transfected can then be used for screening using atomic absorptions spectrometry, as described above.

### Whole-Cell Voltage Clamp Electrophysiology Assay

Electrophysiological recordings of sodium currents in cells stably expressing the voltage-gated sodium channel of interest confirms activity and provides a functional measure of the potency of compounds that specifically affect such channels.

Electrophysiological studies was performed using automated patch-clamp electrophysiology platforms, like IonWorks HT, IonWorks Quattro, PatchXpress, or any other suitable platform. The cell line expressing the voltage-gated sodium channel of interest was plated in appropriate well tissue plates, as provided by the manufacturer of the automated patch-clamp platforms. Suitable extracellular and intracellular buffer for such experiments was applied according to the instructions given by the manufacturer of the automated patch-clamp platforms. Cells that express the voltage-gated sodium channel protein of interest was exposed to drugs through the pipetting system integrated in the platforms. A suitable voltage stimulus protocol was used to activate the voltage-gated sodium channel proteins of interest. A suitable stimulus protocol consisted of eight voltage pulses, each to −20 mV and 50 ms in length, and separated from each other by 330 ms intervals at a potential of −90 mV, or −65 mV.

Electrophysiological studies can also be performed using the whole cell configuration of the standard patch clamp technique as described in the literature (26). In this assay, cells that express the human voltage-gated sodium channel protein of interest are exposed to the drugs by conventional microperfusion systems and a suitable voltage stimulus protocol is used to activate the voltage-gated sodium channels.

Title compounds of the above Examples were tested in whole-cell voltage clamp electrophysiology assay described above and were found to exhibit IC₅₀ values of less than 30 μm, preferably less than 10 μm. The result is presented in following table.

| Compound: | pIC₅₀ |
|---|---|
| N-(2,6-dimethylphenyl)-2-(4-fluoro-2-methoxybenzyl)-3-oxoisoindoline-1- | 7.0 |
| carboxamide | |
| N-(2,6-dimethylphenyl)-2-{2-[2-fluoro-5-(trifluoromethyl)phenoxy]ethyl}-3- | 6.7 |
| oxoisoindoline-1-carboxamide | |
| N-(2,6-dichlorophenyl)-2-(2-methoxybenzyl)-3-oxoisoindoline-1-carboxamide | 6.6 |
| N-(2-chloro-6-methylphenyl)-2-(2-methoxybenzyl)-3-oxoisoindoline-1- | 6.5 |
| carboxamide | |

1. A compound of Formula I 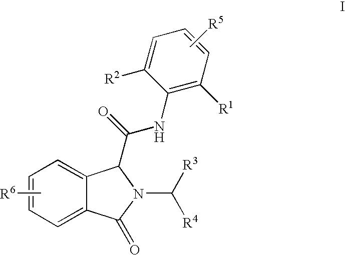
or a pharmaceutically acceptable derivative thereof,
wherein
R¹ and R² represent independently, at each occurrence, halogen, C₁-C₁₂ alkyl, C₁-C₁₂ alkoxy, C₁-C₁₂ haloalkyl, C₁-C₁₂ haloalkoxy, cyano, SR⁷, N(R^{8a})R^{8b}, C2-C6 alkynyl, aryl or Het¹;
R³ represents hydrogen or C₁-C₁₂ alkyl;
R⁴ represents —(CH₂)ₘR⁹ or —(CH₂)ₙOR¹⁰;
further R³ and R⁴ may together represent a ring;
R⁵ represents hydrogen, C₁-C₁₂ alkyl group or C₁-C₁₂ alkoxy group (which C₁-C₁₂ alkyl and C₁-C₁₂ alkoxy groups are optionally substituted by one or more groups selected from halogen, C₂-C₆ alkenyl, C₂-C₆ alkynyl, cyano, oxo, aryl, Het⁴, —OR¹³, —SR¹⁴, —COXR¹⁵, —N(R^{16a})R^{16b}, and —SO₂R¹⁷);
R⁶ represents hydrogen, C₁-C₁₂ alkyl group or C₁-C₁₂ alkoxy group (which C₁-C₁₂ alkyl and C₁-C₁₂ alkoxy groups are optionally substituted by one or more groups selected from halogen, C₂-C₆ alkenyl, C₂-C₆ alkynyl, cyano, oxo, aryl, Het⁵, —OR¹⁸, —SR¹⁹, —COXR²⁰, —N(R^{21a})R^{21b}, —SO₂R²²);
Het¹ to Het⁵ independently represent, at each occurrence, five- to twelve-membered heterocyclic groups containing one or more heteroatoms selected from oxygen, nitrogen and/or sulfur, which groups are optionally substituted by one or more substituents selected from —OH, oxo, halo, cyano, nitro, C₁₋₆ alkyl, C₁₋₆ alkoxy, aryl, aryloxy, —N(R^{23a})R^{23b}, —C(O)R^{23c}, —C(O)OR^{23d}, —C(O)N(R^{23e})R^{23f}, —N(R^{23g})C(O)R^{23h}, —N(R²³ⁱ)S(O)₂R^{23j}, OC(O)R^{23k} and a further Het;
R⁷ and R⁸ represent independently, at each occurrence, hydrogen or C₁₋₆ alkyl;
R⁹ and R¹⁰ represents aryl, Het² (which aryl and Het² optionally are substituted by one or more groups selected from halogen, C₁-C₆ alkyl, C₁-C₆ haloalkyl, C₁-C₆ alkoxy, C₁-C₆ haloalkoxy, cyano, SR¹, N(R^{12a})R^{12b}, C₂-C₆ alkenyl, aryl, and Het³;
R¹¹, R^{12a}, R^{12b}, R¹³, R¹⁴, R¹⁵, R^{16a}, R^{16b}, R¹⁷, R¹⁸, R¹⁹, R²⁰, R^{21a}, R^{21b}, R²², R^{23a}, R^{23b}, R^{23c}, R^{23d}, R^{23e} R^{23f}, R^{23g}, R^{23h} R²³ⁱ, R^{23j} and R^{23k} represent independently, at each occurrence, hydrogen or C₁₋₆ alkyl;
m represents an integer selected from 0, 1, 2 or 3;
n represents an integer selected from 1, 2 or 3;
X represents nitrogen or oxygen atom.

2. A compound according to claim 1 wherein
R¹ and R² represent independently, at each occurrence, halogen, C₁-C₃ alkyl, cyano, SR⁷, N(R^{8a})R^{8b}, C₂-C₃ alkynyl, C₁-C₃ haloalkyl or C₁-C₃ alkoxy;
R³ represents hydrogen or C₁-C₃ alkyl;
R⁴ represents —(CH₂)ₘR⁹ or —(CH₂)ₙOR¹⁰
R³ and R⁴ may together represent a ring;
R⁵ represents hydrogen, C₁-C₃ alkyl (which C₁-C₃ alkyl is optionally substituted by one or more groups selected from halogen, C₂-C₃ alkenyl, C₂-C₃ alkynyl, cyano, —OR¹³, —SR¹⁴, —N(R^{16a})R^{16b});
R⁶ represents hydrogen, C₁-C₃ alkyl or C₁-C₃ alkoxy (which C₁-C₃ alkyl and C₁-C₃ alkoxy are optionally substituted by one or more groups selected from halogen, C₂-C₃ alkenyl, C₂-C₃ alkynyl, cyano, —OR¹⁸, and —N(R^{21a})(R^{21b});
R⁷ and R⁸ represent independently, at each occurrence, hydrogen or C₁₋₃ alkyl;
R⁹ and R¹⁰ represents aryl, Het (which aryl and Het optionally are substituted by one or more groups selected from halogen, C₁-C₆ alkyl, C₁-C₆ haloalkyl, C₁-C₆ alkoxy, C₁-C₆ haloalkoxy, cyano, SR¹¹, N(R^{12a})R^{12b}, C₂-C₆ alkenyl, aryl, and Het²;
R¹¹, R^{12a}, R^{12b}, R¹³, R¹⁴, R¹⁵, R^{16a}, R^{16b}, R¹⁷, R¹⁸, R¹⁹, R²⁰, R^{21a}, R^{21b}, R²², R^{23a}, R^{23b} R^{23c}, R^{23d}, R^{23e} R^{23f}, R^{23g}, R^{23h} R²³ⁱ, R^{23j} and R^{23k} represent independently, at each occurrence, hydrogen or C₁₋₃ alkyl;
m represents 0 or 1;
n represents 1.

3. A compound according to claim 1 wherein
R¹ and R² represent independently, at each occurrence, halogen, or C₁-C₃ alkyl;
R³, R⁵ and R⁶ represent hydrogen;
R⁴ represents —(CH₂)ₘR⁹ or —(CH₂)ₙOR¹⁰;
R⁹ and R¹⁰ represent aryl, Het (which aryl and Het groups optionally are substituted by one or more groups selected from halogen, C₁-C₆ alkyl, C₁-C₆ haloalkyl, C₁-C₆ alkoxy, and C₁-C₆ haloalkoxy);
m represents 0 or 1;
n represents 1.

4. A compound selected from the following compounds:
N-(2,6-dimethylphenyl)-2-(2-ethoxybenzyl)-3-oxoisoindoline-1-carboxamide;
N-(2-chloro-6-methylphenyl)-2-(2-methoxybenzyl)-3-oxoisoindoline-1-carboxamide;
N-(2,6-dichlorophenyl)-6-fluoro-2-(2-methoxybenzyl)-3-oxoisoindoline-1-carboxamide;
2-(2,3-dihydro-1H-inden-1-yl)-N-(2,6-dimethylphenyl)-3-oxoisoindoline-1-carboxamide;
N-(2,6-dimethylphenyl)-2-(2-isopropoxybenzyl)-3-oxoisoindoline-1-carboxamide;
N-(2,6-dimethylphenyl)-6-fluoro-2-(2-methoxybenzyl)-3-oxoisoindoline-1-carboxamide;
N-(2,6-dimethylphenyl)-3-oxo-2-(1,2,3,4-tetrahydronaphthalen-1-yl)isoindoline-1-carboxamide;
N-(2,6-dimethylphenyl)-2-[1-(2-methoxyphenyl)ethyl]-3-oxoisoindoline-1-carboxamide;
N-(2,6-dimethylphenyl)-2-(2-hydroxybenzyl)-3-oxoisoindoline-1-carboxamide;
N-(2,6-dimethylphenyl)-3-oxo-2-[2-(trifluoromethoxy)benzyl]isoindoline-1-carboxamide;
N-(2,6-dimethylphenyl)-2-{2-[(2,6-dimethylpyridin-3-yl)oxy]ethyl}-3-oxoisoindoline-1-carboxamide;
N-(2,6-dimethylphenyl)-2-{2-[4-fluoro-3-(trifluoromethyl)phenoxy]ethyl}-3-oxoisoindoline-1-carboxamide;
N-(2,6-dimethylphenyl)-2-[(2R)-8-methoxy-1,2,3,4-tetrahydronaphthalen-2-yl]-3-oxoisoindoline-1-carboxamide;
N-(2,6-dimethylphenyl)-2-{1-methyl-2-[3-(trifluoromethyl)phenoxy]ethyl}-3-oxoisoindoline-1-carboxamide;
N-(2,6-dimethylphenyl)-2-{2-[2-fluoro-5-(trifluoromethyl)phenoxy]ethyl}-3-oxoisoindoline-1-carboxamide;
N-(2,6-dichlorophenyl)-2-(2-methoxybenzyl)-3-oxoisoindoline-1-carboxamide;
2-(2,3-dihydro-1,4-benzodioxin-2-ylmethyl)-N-(2,6-dimethylphenyl)-3-oxoisoindoline-1-carboxamide;
N-(2,6-dimethylphenyl)-2-[(2S)-8-methoxy-1,2,3,4-tetrahydronaphthalen-2-yl]-3-oxoisoindoline-1-carboxamide;
N-(2,6-dimethylphenyl)-2-[(3S)-5-methoxy-3,4-dihydro-2H-chromen-3-yl]-3-oxoisoindoline-1-carboxamide;
N-(2,6-dimethylphenyl)-3-oxo-2-[(1R)-1-phenylethyl]isoindoline-1-carboxamide;
N-(2,6-dimethylphenyl)-2-(2-methoxybenzyl)-3-oxoisoindoline-1-carboxamide;
N-(2,6-dimethylphenyl)-2-(2-methylbenzyl)-3-oxoisoindoline-1-carboxamide;
2-[2-(4-chlorophenyl)propyl]-N-(2,6-dimethylphenyl)-3-oxoisoindoline-1-carboxamide;
2-(biphenyl-2-ylmethyl)-N-(2,6-dimethylphenyl)-3-oxoisoindoline-1-carboxamide;
N-(2-isopropyl-6-methylphenyl)-2-(2-methoxybenzyl)-3-oxoisoindoline-1-carboxamide;
2-(2,4-difluorobenzyl)-N-(2-isopropyl-6-methylphenyl)-3-oxoisoindoline-1-carboxamide;
N-(2-isopropyl-6-methylphenyl)-2-[(3-methylpyridin-2-yl)methyl]-3-oxoisoindoline-1-carboxamide;
2-(2-fluorobenzyl)-N-mesityl-3-oxoisoindoline-1-carboxamide;
N-(2,6-dimethylphenyl)-3-oxo-2-(pyridin-2-ylmethyl)isoindoline-1-carboxamide;
2-(3-chlorobenzyl)-N-(2-isopropyl-6-methylphenyl)-3-oxoisoindoline-1-carboxamide;
2-(3-chlorobenzyl)-N-mesityl-3-oxoisoindoline-1-carboxamide;
N-(2,6-dimethylphenyl)-2-[2-(7-methyl-1H-indol-3-yl)ethyl]-3-oxoisoindoline-1-carboxamide;
2-(2,5-dimethoxybenzyl)-N-(2,6-dimethylphenyl)-3-oxoisoindoline-1-carboxamide;
N-(2-chloro-6-methylphenyl)-2-[2-(3-methoxyphenyl)ethyl]-3-oxoisoindoline-1-carboxamide;
N-(2,6-dimethylphenyl)-2-(2-methoxybenzyl)-N-methyl-3-oxoisoindoline-1-carboxamide;
N-(2,6-dimethoxyphenyl)-2-(2-methoxybenzyl)-3-oxoisoindoline-1-carboxamide;
N-(2,6-dimethylphenyl)-2-(4-fluoro-2-methoxybenzyl)-5-hydroxy-4-methyl-3-oxoisoindoline-1-carboxamide; and
N-(2,6-dimethylphenyl)-2-(4-fluoro-2-methoxybenzyl)-3-oxoisoindoline-1-carboxamide;
or pharmaceutically acceptable derivative thereof.

5-6. (canceled)

7. A pharmaceutical composition comprising as active ingredient a therapeutically effective amount of the compound according to claim 1, in association with one or more pharmaceutically acceptable diluents, excipients and/or inert carriers.

8. (canceled)

9. A method of treatment of chronic, acute, neuropathic, nociceptive, visceral or inflammatory pain, comprising administering to a mammal, including man in need of such treatment, a therapeutically effective amount of a compound according to claim 1.

10. (canceled)
